(19) United States
(12) Patent Application Publication
(10) Pub. No.: US 2024/0082490 A1
(43) Pub. Date: Mar. 14, 2024

(54) APPARATUS AND METHOD FOR REMOVING GAS FROM AN INFUSION LINE

(71) Applicant: CareFusion 303, Inc., San Diego, CA (US)

(72) Inventors: Frank CAI, Ontario, CA (US); Derek Alan CARROLL, Carson, CA (US); Siddarth SHEVGOOR, Mission Viejo, CA (US); Lun ZHANG, Brea, CA (US)

(21) Appl. No.: 18/307,767

(22) Filed: Apr. 26, 2023

Related U.S. Application Data
(60) Provisional application No. 63/336,215, filed on Apr. 28, 2022.

(51) Int. Cl. A61M 5/165

(52) U.S. Cl. CPC A61M 5/165; A61M 2005/1657

(57) ABSTRACT

An infusion line gas removal device and process for its fabrication and use is disclosed herein. The gas removal device includes a gas permeable tubing coupled between first and second couplers, and an impermeable tubing shield encasing the gas permeable tubing between the first and second couplers. The impermeable tubing shield is larger in diameter than the gas permeable tubing and comprises at least one vent and a plurality of supports, the plurality of supports being between the tubing shield and the gas permeable tubing and configured to support the gas permeable tubing within the impermeable tubing shield in a fixed position between the first and second couplers and facilitate passage of gas from the gas permeable tubing to the at least one vent.

### CROSS-REFERENCE TO RELATED APPLICATIONS

This application claims the benefit of U.S. Provisional Application No. 63/336,215, filed on Apr. 28, 2022, the entirety of each of which is incorporated herein by reference for all purposes.

### BACKGROUND

There are a variety of situations where fluid is infused to a patient. Applications of fluid delivery systems include (but are by no means limited to) intravenous infusion, intra-arterial infusion, infusion of enteral solutions, infusion of medication to the epidural space, and diagnostic infusion to determine vascular characteristics of the arterial, urinary, lymphatic, or cerebrospinal systems.

Fluid delivery systems for infusing fluid to a patient typically include a supply of the fluid to be administered, an infusion needle or cannula, an administration set connecting the fluid supply to the cannula, and a flow control device, such as a positive displacement infusion pump. The administration set typically comprises a length of flexible tubing. The cannula is mounted at the distal end of the flexible tubing for insertion into a patient's blood vessel or other body location to deliver the fluid infusate to the patient.

During an infusion procedure, various agents, the most typical of which is air, can be introduced into the fluid delivery system by a number of events, including the fluid supply becoming drained of fluid. Because introducing excessive air into the patient's blood system may create complications. In cases where the infusion is left unattended and enough air is infused into the patient, serious injury may occur.

### SUMMARY

According to various aspects of the subject technology, an infusion line gas removal device, comprises a gas permeable tubing coupled between first and second couplers; and an impermeable tubing shield encasing the gas permeable tubing between the first and second couplers, wherein the impermeable tubing shield is larger in diameter than the gas permeable tubing and comprises at least one vent and a plurality of supports, the plurality of supports being between the tubing shield and the gas permeable tubing and configured to support the gas permeable tubing within the impermeable tubing shield in a fixed position between the first and second couplers and facilitate passage of gas from the gas permeable tubing to the at least one vent. Other aspects include corresponding systems, methods and processes for implementation of the corresponding gas removal device.

According to various aspects of the subject technology, a process of providing an infusion line gas removal device, comprising providing a gas permeable tubing coupled between first and second couplers; and encasing the gas permeable tubing with an impermeable tubing shield between the first and second couplers, wherein the impermeable tubing shield is larger in diameter than the gas permeable tubing and comprises at least one vent and a plurality of supports, the plurality of supports being between the tubing shield and the gas permeable tubing and configured to support the gas permeable tubing within the impermeable tubing shield in a fixed position between the first and second couplers and facilitate passage of gas from the gas permeable tubing to the at least one vent.

According to various aspects of the subject technology, a method for removal of gas from an infusion line comprises providing a gas permeable tubing coupled between first and second couplers; and providing an impermeable tubing shield encasing the gas permeable tubing between the first and second couplers, wherein the impermeable tubing shield is larger in diameter than the gas permeable tubing and comprises at least one vent and a plurality of supports, the plurality of supports being between the tubing shield and the gas permeable tubing and configured to support the gas permeable tubing within the impermeable tubing shield in a fixed position between the first and second couplers and facilitate passage of gas from the gas permeable tubing to the at least one vent.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example infusion pump set-up, shown in use in its intended environment, according to various aspects of the subject technology.

FIG. 2 depicts an example infusion line gas removal device, according to various aspects of the subject technology.

FIG. 3 is an exploded depiction of the example infusion line gas removal device, according to various aspects of the subject technology.

FIGS. 4A and 4B depict a second example infusion line gas removal device, according to various aspects of the subject technology.

FIGS. 5A and 5B depict an example infusion line gas removal device including a connected vacuum source, according to various aspects of the subject technology.

FIGS. 6A and 6B depict example alternative configurations of a gas permeable tubing, according to various aspects of the subject technology.

FIG. 7 depicts a first example process for removing gas from an infusion line, according to various implementations of the subject technology.

FIG. 8 depicts a second example process for fabricating or otherwise providing an infusion line gas removal device, according to various implementations of the subject technology.

### DETAILED DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

The disclosed infusion line gas removal device includes a disposable apparatus that can be connected onto the distal end of an infusion pump set to trap air present in the infusion line and to expel it through an air permeable tubing. A tubing expansion shield encases the tubing to prevent expansion, particularly in instances where the gas permeable tubing may be soft and pliable, and prevent failure in the event of a pressure spike. The tubing expansion shield may have many gaps to allow air to escape from the tubing. At the edges of the tubing expansion shield are air vents with filters to allow air to escape while preventing any airborne particulate from entering. A bubble diverter is included in some implementations and attached at the distal end of the device to direct bubbles towards the tubing wall, making it difficult for air bubbles to pass through and continue toward the patient.

In some implementations, the device is coupled with an anti-siphon valve upstream of the air removal device, which helps maintain positive pressure below the valve in the event of momentary retrograde flow. This valve facilitates a constant pressure differential, with the pressure within the gas permeable tubing being higher than atmospheric pressure. In some implementations, a vacutainer replaces and/or is coupled to the air vents (e.g., with a spike) and pulls air out of the gas permeable tubing. A small membrane within the vacutainer serves as an indicator of the presence of vacuum.

Advantages of the disclosed infusion line gas removal device (over traditional air removal technologies and devices) include that it may be configured anywhere in an infusion line between an infusion fluid source and/or the pump and a patient, and may be oriented in any direction and/or is not subject to diminished performance due to different or changing orientations during an infusion.

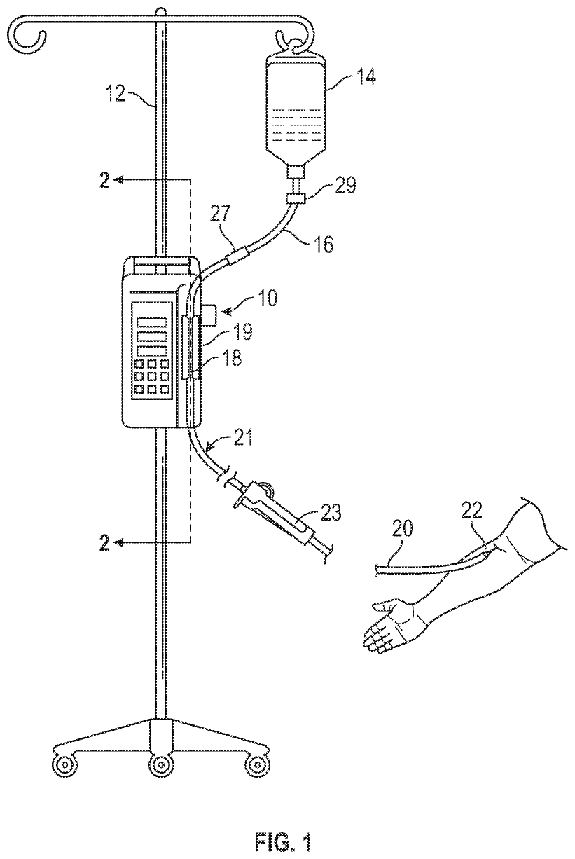

FIG. 1 depicts an example infusion pump set-up 10, shown in use in its intended environment, according to various aspects of the subject technology. In particular, the infusion pump set-up 10 is shown mounted to an intravenous (IV) pole 12 on which a fluid source 14 containing an IV fluid is held. The fluid source 14 is connected in fluid communication with an upstream fluid line 16. The fluid line 16 is a conventional IV infusion type tube typically used in a hospital or medical environment, and is made of any type of flexible tubing appropriate for use to infuse therapeutic fluids into a patient, such as polyvinylchloride (PVC). A flexible pumping fluid line 18 is mounted in operative engagement with a peristaltic pumping apparatus 19, for propelling fluid through a downstream fluid line 20, for example, to a patient's arm. A cannula 22 is mounted at the distal end of the flexible IV tubing 21 for insertion into a patient's blood vessel or other body location to deliver the fluid to the patient.

It will be understood by those skilled in the art that the upstream fluid line 16, the flexible line 18, and the downstream fluid line 20 may be portions of a continuous length of flexible tubing, with the portions defined by the location of the peristaltic pump 19. For convenience, the continuous length of flexible tubing is indicated by numeral 21. A roller clamp 23 (e.g., configured to provide for mechanical compression of the line to block the flow) may be positioned on the downstream fluid line 20 between the pump 10 and the patient's arm 22. In this context, the term “upstream” refers to that portion of the flexible tubing that extends between the fluid source and peristaltic pump, and the term “downstream” refers to that portion of the flexible tubing that extends from the peristaltic pump to the patient.

Also shown in FIG. 1 is a secondary administration setup generally indicated by numeral 24. The secondary administration setup 24 includes a secondary fluid container 25 that may be filled with a second therapeutic fluid for infusion into the patient 22. Fluid from the secondary fluid container 25 flows through a secondary fluid line 26 into the fluid line 16 through a connector 27. A manually operated valve 28 is located in the secondary line 26 to control the flow of fluid flowing out of the secondary container 25 into the upstream fluid line 16. The one-way check valve 29 is disposed in the upstream fluid line 16 between the primary fluid container 14 and the connector 27, the one-way check valve is configured so that when the elevation of the fluid in the secondary container 25 is greater than that of the primary container, the differential pressure within line 16 closes the check valve and prevents secondary fluid from flowing into the primary container 14, and also prevents fluid from flowing out of primary container 14. Thus, the check valve 29 generally prevents mixing of the primary and secondary infusion fluids.

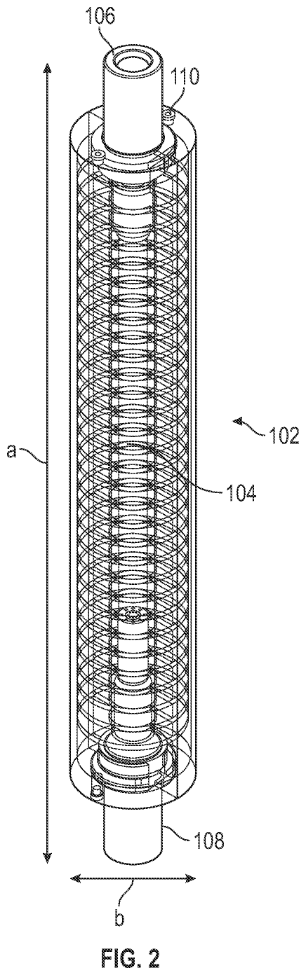

FIG. 2 depicts an example infusion line gas removal device 100, according to various aspects of the subject technology. The disclosed gas removal device 100 may be made of disposable materials (e.g., plastic, PVC, silicon, etc.), and typically has length a that is substantially greater than its diameter b. The gas removal device 100 is configured to be connected onto the distal end of an infusion pump set, for example onto downstream fluid line 20, before cannula 22. For example, fluid line 20 may comprise an upstream and downstream portions to which the gas removal device 100 may be connected between.

As will be described further, gas removal device 100 is configured to trap air present in fluid line 20 and expel it. A tubing expansion shield 102 encases a gas permeable tubing 104 coupled between first and second couplers 106, 108 to facilitate removal of gas within fluid line 20 as fluid passes through the device 100 while preventing expansion of the tubing 104 encased within expansion shield 102. According to various implementations, tubing expansion shield constrains the tubing 104 while allowing gas to escape from the tubing within the shield 102. A gas, as termed in this description, includes one or more elemental gases and/or gas mixtures such as air.

At the edges of the tubing expansion shield are air vents 110 configured to allow air to escape. Filters 110-a may be integrated into the vents 110 to prevent any airborne particulate from entering the device. A bubble diverter 112 may also be included and attached at the distal end of the device to direct bubbles towards the tubing wall and make it difficult for air bubbles to pass through and continue toward the patient.

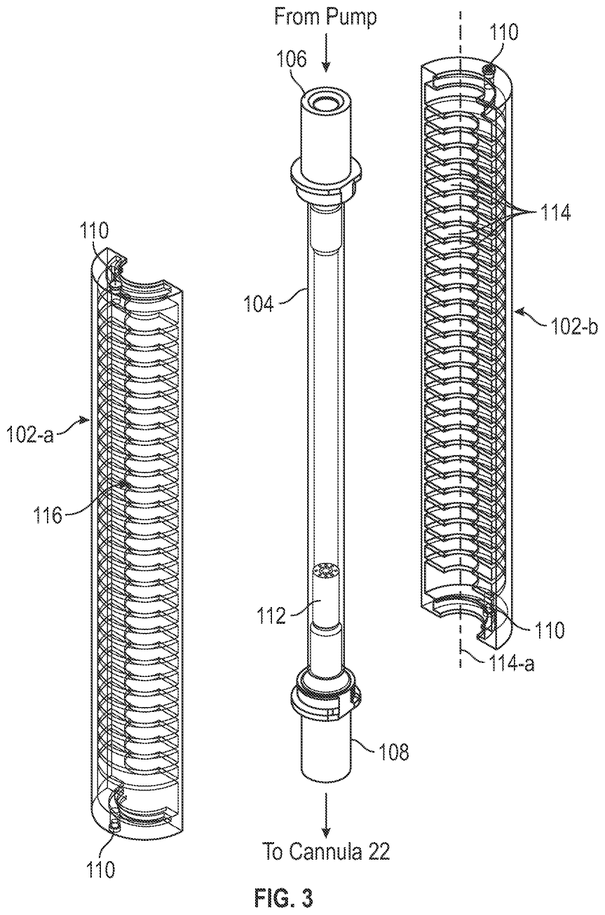

FIG. 3 is an exploded depiction of the example infusion line gas removal device, according to various aspects of the subject technology. According to various implementations, the infusion line gas removal device 100 includes a gas permeable tubing 104 coupled between first and second couplers 106, 108. An impermeable tubing shield 102, when assembled, encases the gas permeable tubing 104 between the first and second couplers 106, 108. The gas permeable tubing 104 may be formed of a membrane that has gas permeable capabilities. The membrane may be configured such that a pressure delta between fluid in the tubing and the atmospheric temperature will drive air through the membrane and outside the tubing. However, the pores of the membrane are too small to allow fluid to pass through. According to various implementations, the amount of gas removed during an infusion is a function of the overall length of the fluid path (e.g., a), cross sectional area of the tubing 104, cross sectional area of shield 102

$$( e . g . , {π ( \frac{b}{2} )}^{2} ) ,$$

flow rate, fluid type, and permeability of the membrane.

As depicted in FIGS. 2 and 3, the impermeable tubing shield 102 is impermeable to gases such as air and larger in diameter than the gas permeable tubing 104. The shield 102 includes one or more vents 110 and a plurality of supports 114. According to various implementations, the plurality of supports 114 are located within the shield, between the tubing shield 102 and the gas permeable tubing 104, and are configured to support the gas permeable tubing 104 within the impermeable tubing shield 102 in a fixed position between the first and second couplers 106, 108. According to various implementations, the supports 114 are equally spaced apart from each other, providing many gaps to facilitate passage of gas from the gas permeable tubing 104 to at least one vent 110.

According to various implementations, the plurality of supports 114 are configured to support the gas permeable tubing 104 within the impermeable tubing shield 102 by being periodically positioned to provide support to an exterior of the gas permeable tubing 102 to prevent a pressure spike within the gas permeable tubing from causing an expansion of the gas permeable tubing 102. In this regard, each of the supports 114 may include, when the device is formed, at least a portion of a disc with an aperture 114-a at its center that at least partially encases and provides support to a portion of the gas permeable tubing 104 while facilitating the passage of the gas from the gas permeable tubing to the at least one vent.

As depicted in FIG. 3, the impermeable tubing shield 102 may be formed from two molded cross sections 102-a and 102-b, with each cross section traversing a length of the gas permeable tubing 102. The supports 114, when the two molded cross sections are coupled together around the gas permeable tubing 104, come together to form a complete array of supports 114, as depicted, and support the gas permeable tubing 104 within the impermeable tubing shield in the fixed position. The cross sections 102-ab may be injection molded and then coupled (e.g., with adhesive) around/over the gas permeable tubing. In some implementations, the tubing may be inserted into and through a formed tubing shield 102.

According to various implementations, the supports 114 are evenly spaced such to provide uniform support to the gas permeable tubing 104 therein. The supports 114 may each be, when the device is completed, a disc with an aperture 114-a at its center that constrains a portion of the gas permeable tubing. In some implementations, the supports may be a portion of a disc. In some implementations, the supports may take on a different shape or combination of shapes. In the depicted implementation, the device 100 is in the shape of a cylinder and the discs conform to the shape of the cylinder. In implementations wherein the external cross-sectional shape of the shield 102 is not circular (e.g., ovoid or rectangular), the supports may be shaped to conform to the shape of the shield. Similarly, when the cross sectional shape of the tubing is non-circular (e.g., ovoid or rectangular or star-shaped) the aperture 114-a may be shaped according to the cross sectional shape of the tubing 104.

In some implementations, the impermeable tubing shield 102 includes a gas channel 116 whereby gas may enter via the spaces between the supports 114 and be directed toward the vents 110. As depicted, the gas channel 116 may be made up by a linear alignment of gaps in an outer edge of each support 114, for example, between impermeable tubing shield and the disc, at the outer edge of the disc. In some implementations, each gap of the plurality of supports may be aligned with the at least one vent. In some implementations, the gaps may be staggered to calibrate the passage of air from the tubing 104 to the vent(s) 110.

In some implementations, as depicted in FIG. 3, the infusion line air removal device 100 includes a bubble diverter 118 coupled to one of the first and second couplers 106, 108 within the gas permeable tubing 104. For example, the bubble diverter 118 may be coupled to the downstream coupler 108 and inserted in the downstream section of the tubing 104 with a portion of coupler 108 inserted into the same downstream section of tubing 104. The bubble diverter 118 configured to control the position of air bubbles in a fluid path by taking advantage of the surface tension of an air bubble. For example, the diverter 118 directs bubbles within a fluid flowing inside the gas permeable tubing (around the diverter) toward a wall of the gas permeable tubing.

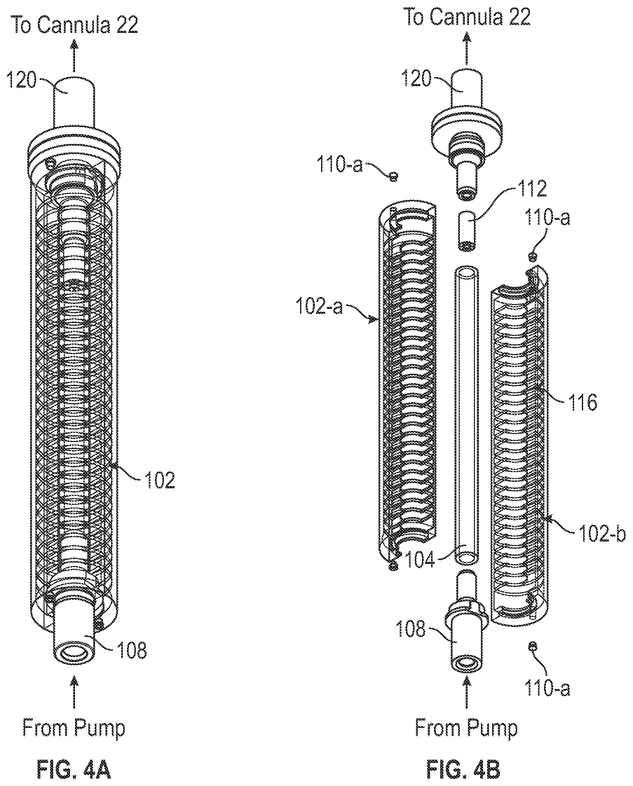

FIGS. 4A and 4B depict a second example infusion line gas removal device, according to various aspects of the subject technology. The depicted device is fashioned as previously described. In the depicted implementation, the upstream coupler 106 functions as or is replaced by an anti-siphon valve 120. The anti-siphon valve 120 is configured to facilitate fluid moving in only one direction and to facilitate positive pressure within the gas permeable tubing. While the valve 120 is depicted in the upstream position, the device 100 may be fabricated with the valve in the downstream position.

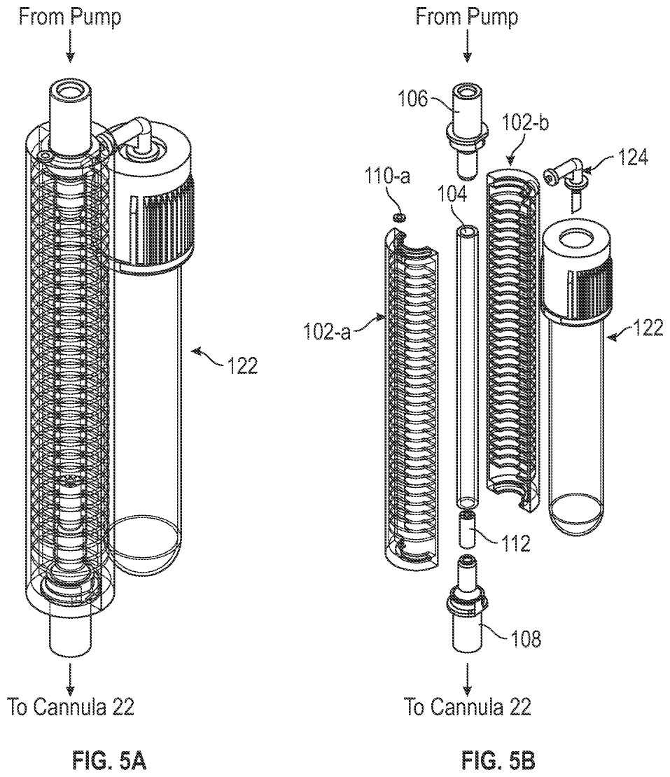

FIGS. 5A and 5B depict an example infusion line gas removal device including a connected vacuum source 122, according to various aspects of the subject technology. According to various implementations, the vacuum source includes a vacutainer; i.e., a vacuum tube or container with a seal or stopper that creates a vacuum seal inside the tube or container. The vacuum source 122 is fluidically sealed to the at least one vent 110 and creates a vacuum within the tubing shield 102, facilitating removal of gas (through the gas permeable tubing 104) from a fluid flowing inside the gas permeable tubing 104. For example, the vacuum may actively pull air out of the gas permeable tubing.

As in the depicted example, a vacutainer 122 may be secured to a vent 110 by way of a conduit 124 having a spike inserted into the vacutainer. The conduit may be formed as an elbow conduit 124 so that the vacutainer is aligned and/or parallel with the device 100. In some implementations (not shown), instead of a vacutainer or similar device, an active vacuum line may be attached to the vent 110. In some implementations, the vacuum source 122 includes a flexible membrane (e.g., within the vacutainer) configured to flex and become concave within the flexible membrane in response to the vacuum.

In some implementations, use of vacuum source 122 may obviate a need for the anti-siphon valve 120 described with regard to FIGS. 4A and 4B. Both may be used together but at an increased cost per unit.

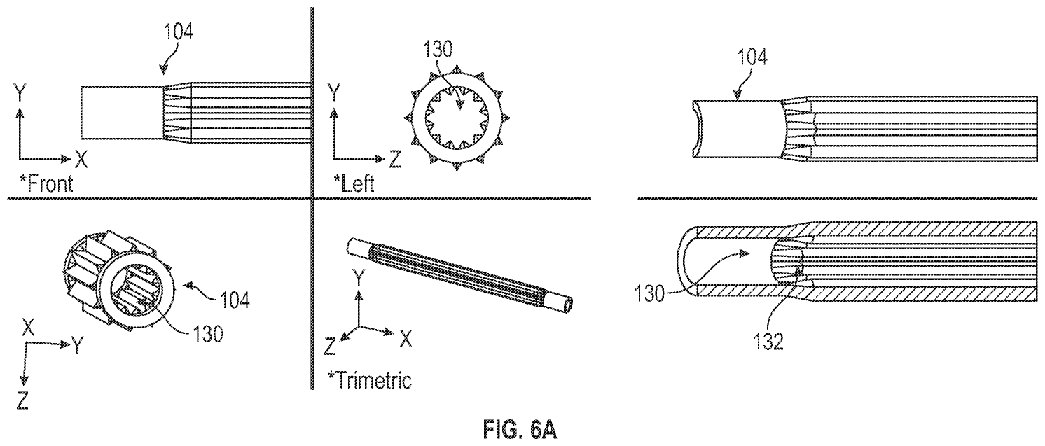

FIGS. 6A and 6B depict example alternative configurations of a gas permeable tubing, according to various aspects of the subject technology. Different tubing geometries may be employed to maximize the amount of air permeating through the tubing wall of tubing 104. FIG. 6A depicts the gas permeable tubing 104 having a pleated interior surface 130 with a plurality of ridges 132 formed along a length of the gas permeable tubing (e.g., similar to that seen in a pleated filter). Accordingly, the surface 130 of the tubing depicted in FIG. 6A has a larger surface area than the tubing depicted in FIGS. 2-5, even if having the same diameter without the ridges 132.

FIG. 6B depicts the gas permeable tubing 104 having a circular cross section 134 at the first and second couplers 106, 108 and an ovoid cross section 136 between the first and second couplers. According to various implementations, the cross section of the tubing 104 may be rectangular. The impermeable tubing shield 102 may be modified to accommodate different variations of the tubing 104. For example, the tubing shield 102 may have an ovoid or rectangular cross section. In some implementations, the tubing shield 102 may have an ovoid or rectangular cross section while the gas permeable tubing has a circular cross section.

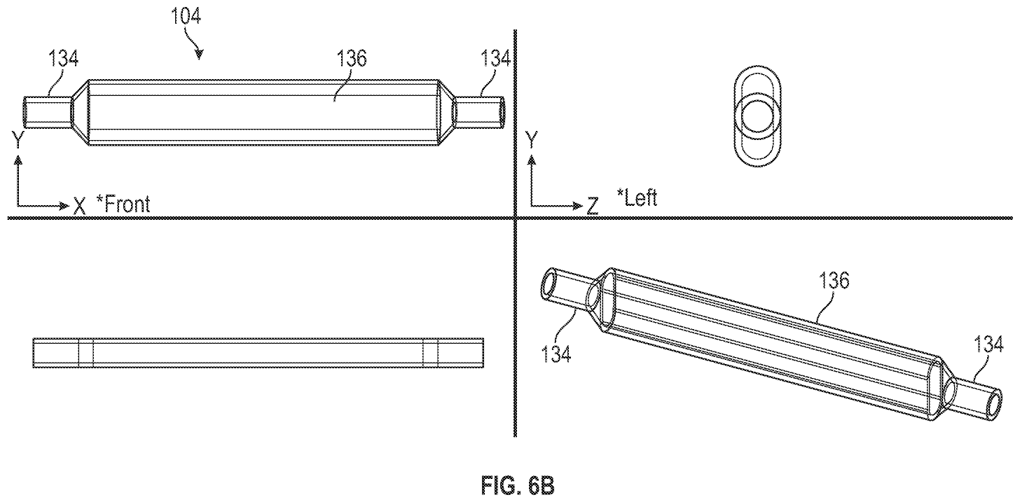

FIG. 7 depicts a first example process for removing gas from an infusion line, according to various implementations of the subject technology. For explanatory purposes, the various blocks of example process 200 are described herein with reference to FIGS. 1-6, and the components and/or processes described herein. In some implementations, one or more of the blocks may be implemented apart from other blocks, and by one or more different devices. Further for explanatory purposes, the blocks of example process 200 are described as occurring in serial, or linearly. However, multiple blocks of example process 200 may occur in parallel. In addition, the blocks of example process 200 need not be performed in the order shown and/or one or more of the blocks of example process 200 need not be performed.

In the depicted example, a gas permeable tubing 104 coupled between first and second couplers is provided (202). An impermeable tubing shield 102 encasing the gas permeable tubing 104 between the first and second couplers is also provided (204). According to various implementations, the impermeable tubing shield 102 is larger in diameter than the gas permeable tubing 104 and comprises at least one vent 110 and a plurality of supports 114. The plurality of supports 114 are positioned between the tubing shield 102 and the gas permeable tubing 104 and configured to support the gas permeable tubing within the impermeable tubing shield 102 in a fixed position between the first and second couplers 106, 108, and to facilitate passage of gas from the gas permeable tubing 104 to the at least one vent 110, as described with regard to any of the implementations previously described with regard to FIGS. 2-6.

The process optionally further continues by providing a vacuum source configured to be fluidically sealed to the at least one vent (206). According to various implementations, the vacuum source creates a vacuum within the impermeable tubing shield, facilitating removal of the gas from a fluid flowing inside the gas permeable tubing. The vacuum source may include, as described previously, a vacutainer 122 or other tub/container device with a flexible membrane configured to flex and become concave within the flexible membrane in response to the vacuum.

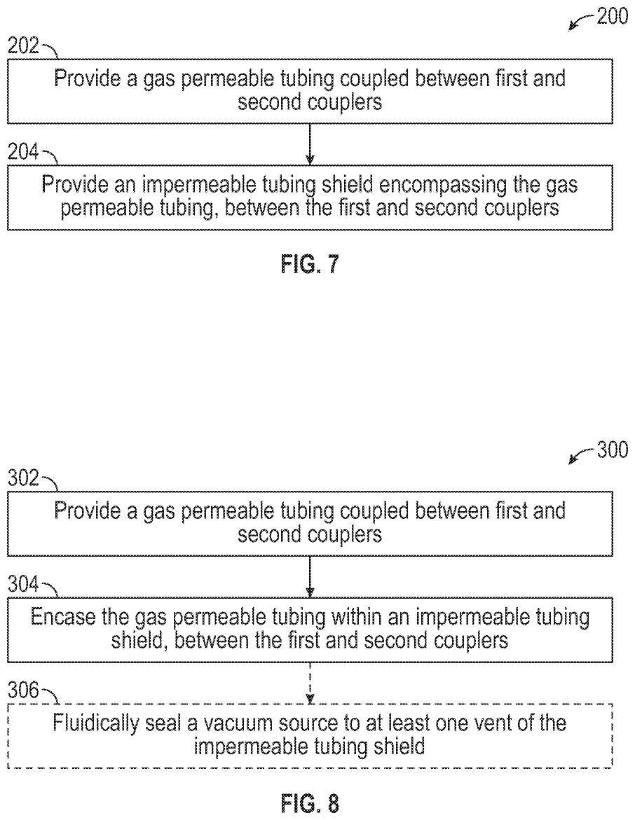

FIG. 8 depicts a second example process for fabricating or otherwise providing an infusion line gas removal device, according to various implementations of the subject technology. For explanatory purposes, the various blocks of example process 300 are described herein with reference to FIGS. 1-7, and the components and/or processes described herein. In some implementations, one or more of the blocks may be implemented apart from other blocks, and by one or more different devices. Further for explanatory purposes, the blocks of example process 300 are described as occurring in serial, or linearly. However, multiple blocks of example process 300 may occur in parallel. In addition, the blocks of example process 300 need not be performed in the order shown and/or one or more of the blocks of example process 300 need not be performed.

In the depicted example, a gas permeable tubing 104 is provided, and coupled between first and second couplers (302). In some implementations, the tubing 104 is of a standard size used in IV infusions. In some implementations, different tubing geometries may be employed at maximize the amount of air permeating through the tubing wall. One such geometry includes the tubing cross section being in the shape of a star, similar to that seen in a pleated filter. Another tubing geometry may be a tubing molded with an oval cross section.

The gas permeable tubing 104 is encased (e.g., enclosed or encompassed) within an impermeable tubing shield 102 between the first and second couplers (304). According to various implementations, the shield 102 may be cylindrical. In some implementations, the shield 102 may be straight (as depicted in FIGS. 2-6). In some implementations, the shield 102 (and the tubing within the shield) may be curved or angled, and/or have multiple curves or angles.

According to various implementations, the impermeable tubing shield 102 is larger in diameter than the gas permeable tubing 104 and comprises at least one vent 110 and a plurality of supports 114. The plurality of supports 114 are between the tubing shield 104 and the gas permeable tubing 102 and configured to support the gas permeable tubing 104 within the impermeable tubing shield 102 in a fixed position between the first and second couplers 106, 108 and facilitate passage of gas from the gas permeable tubing 104 to the at least one vent 110. As described previously, the supports may be formed within the impermeable tubing shield 104 by positioning the plurality of supports 114 in a manner to provide support to an exterior of the gas permeable tubing 104 while preventing pressure spikes within the gas permeable tubing from causing an expansion of the gas permeable tubing.

In accordance with FIG. 3, forming the plurality of supports 114 within the impermeable tubing shield 102 may include forming the impermeable tubing shield 102 in two molded cross sections 102-1, 102-b. In such implementations, each cross section may traverse a length of a gas permeable tubing 104 and include a plurality of partial supports that, when the two molded cross sections are coupled together around the gas permeable tubing (e.g., by adhesive), come together to form the plurality of supports 114 and support the gas permeable tubing 104 within the impermeable tubing shield 102 in the fixed position. In such implementations, the process includes coupling the two molded cross sections 102-1, 102-b around the gas permeable tubing 104.

As described previously, the impermeable tubing shield 102, when formed, may have a circular cross section. In some implementations, the plurality of supports 114, when formed, each may be or include a disc with an aperture 114-a at its center that constrains a portion of the gas permeable tubing. The supports are configured to facilitate the passage of the gas from the gas permeable tubing 104 to the at least one vent 110, the disc coupled to the impermeable tubing shield 102.

According to various implementations, the process optionally continues by fluidically sealing a vacuum source to the at least one vent (306). The vacuum source creates a vacuum within the impermeable tubing shield, facilitating removal of the gas from a fluid flowing inside the gas permeable tubing. The vacuum source may be implemented by any vacuum device or method described herein.

Advantages of the disclosed gas removal device include the ability to be molded and produced at high volumes, thereby driving down cost, while requiring little to no additional clinician training in the field. Furthermore, the device is configured such that it does not change priming volume (e.g., if length of tubing similar to length of device is replaced), and requires no external power source to operate.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. An infusion line gas removal device, comprising: a gas permeable tubing coupled between first and second couplers; and an impermeable tubing shield encasing the gas permeable tubing between the first and second couplers, wherein the impermeable tubing shield is larger in diameter than the gas permeable tubing and comprises at least one vent and a plurality of supports, the plurality of supports being between the tubing shield and the gas permeable tubing and configured to support the gas permeable tubing within the impermeable tubing shield in a fixed position between the first and second couplers and facilitate passage of gas from the gas permeable tubing to the at least one vent.

Clause 2. The infusion line gas removal device of Clause 1, wherein the plurality of supports being configured to support the gas permeable tubing within the impermeable tubing shield in a fixed position comprises the plurality of supports being positioned to provide support to an exterior of the gas permeable tubing to prevent a pressure spike within the gas permeable tubing from causing an expansion of the gas permeable tubing.

Clause 3. The infusion line gas removal device of Clause 2, wherein each of the plurality of supports comprises at least a portion of a disc with an aperture at its center that at least partially encases and provides support to a portion of the gas permeable tubing while facilitating passage of the gas from the gas permeable tubing to the at least one vent, the disc coupled to the impermeable tubing shield.

Clause 4. The infusion line gas removal device of Clause 3, wherein each of the plurality of supports comprises a gap in an outer edge of the disc between the impermeable tubing shield and the disc.

Clause 5. The infusion line gas removal device of Clause 4, wherein each gap of the plurality of supports is aligned with the at least one vent.

Clause 6. The infusion line gas removal device of any one of Clauses 1 to 5, further comprising: a bubble diverter coupled to one of the first and second couplers within the gas permeable tubing, the bubble diverter configured to direct bubbles within a fluid flowing inside the gas permeable tubing toward a wall of the gas permeable tubing.

Clause 7. The infusion line gas removal device of any one of Clauses 1 to 6, wherein the impermeable tubing shield has an ovoid cross section and the gas permeable tubing has a circular cross section.

Clause 8. The infusion line gas removal device of any one of Clauses 1 to 7, wherein one of the first and second couplers comprises an anti-siphon valve configured to facilitate fluid moving in only one direction and to facilitate positive pressure within the gas permeable tubing.

Clause 9. The infusion line gas removal device of any one of Clauses 1 to 8, further comprising: a vacuum source fluidically sealed to the at least one vent, wherein the vacuum source creates a vacuum within the impermeable tubing shield, facilitating removal of the gas from a fluid flowing inside the gas permeable tubing.

Clause 10. The infusion line gas removal device of Clause 9, wherein the vacuum source comprises: a vacutainer connected to the at least one vent; and a flexible membrane, within the vacutainer, configured to flex and become concave within the flexible membrane in response to the vacuum.

Clause 11. The infusion line gas removal device of any one of Clauses 1 to 10, wherein the gas permeable tubing comprises: a pleated interior surface with a plurality of ridges formed along a length of the gas permeable tubing and having a larger surface area than a tubing of a same diameter as the gas permeable tubing.

Clause 12. The infusion line gas removal device of any one of Clauses 1 to 11, wherein the gas permeable tubing has a circular cross section at the first and second couplers and an ovoid cross section between the first and second couplers.

Clause 13. A process of providing an infusion line gas removal device, comprising: providing a gas permeable tubing coupled between first and second couplers; and encasing the gas permeable tubing with an impermeable tubing shield between the first and second couplers, wherein the impermeable tubing shield is larger in diameter than the gas permeable tubing and comprises at least one vent and a plurality of supports, the plurality of supports being between the tubing shield and the gas permeable tubing and configured to support the gas permeable tubing within the impermeable tubing shield in a fixed position between the first and second couplers and facilitate passage of gas from the gas permeable tubing to the at least one vent.

Clause 14. The process of Clause 13, further comprising: forming the plurality of supports within the impermeable tubing shield by positioning the plurality of supports in a manner to provide support to an exterior of the gas permeable tubing to prevent a pressure spike within the gas permeable tubing from causing an expansion of the gas permeable tubing.

Clause 15. The process of Clause 14, wherein forming the plurality of supports within the impermeable tubing shield comprises: forming the impermeable tubing shield in two molded cross sections, each cross section traversing a length of a gas permeable tubing and comprising a plurality of partial supports that, when the two molded cross sections are coupled together around the gas permeable tubing, come together to form the plurality of supports and support the gas permeable tubing within the impermeable tubing shield in the fixed position; and coupling the two molded cross sections around the gas permeable tubing.

Clause 16. The process of Clause 15, wherein the impermeable tubing shield, when formed, has a circular cross section, and the plurality of supports, when formed, each comprise a disc with an aperture at its center that constrains a portion of the gas permeable tubing.

Clause 17. The process of Clause 14 or Clause 15, wherein each of the plurality of supports comprises at least a portion of a disc with an aperture at its center that at least partially encases and provides support to a portion of the gas permeable tubing while facilitating passage of the gas from the gas permeable tubing to the at least one vent, the disc coupled to the impermeable tubing shield.

Clause 18. The process of any one of Clauses 13 to 17, further comprising: fluidically sealing a vacuum source to the at least one vent, wherein the vacuum source creates a vacuum within the impermeable tubing shield, facilitating removal of the gas from a fluid flowing inside the gas permeable tubing.

Clause 19. A method for removal of gas from an infusion line, comprising: providing a gas permeable tubing coupled between first and second couplers; and providing an impermeable tubing shield encasing the gas permeable tubing between the first and second couplers, wherein the impermeable tubing shield is larger in diameter than the gas permeable tubing and comprises at least one vent and a plurality of supports, the plurality of supports being between the tubing shield and the gas permeable tubing and configured to support the gas permeable tubing within the impermeable tubing shield in a fixed position between the first and second couplers and facilitate passage of gas from the gas permeable tubing to the at least one vent.

Clause 20. The method of Clause 19, further comprising: providing a vacuum source configured to be fluidically sealed to the at least one vent, wherein the vacuum source creates a vacuum within the impermeable tubing shield, facilitating removal of the gas from a fluid flowing inside the gas permeable tubing, and includes a flexible membrane configured to flex and become concave within the flexible membrane in response to the vacuum.

### Further Consideration:

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein, the terms “determine” or “determining” encase a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encase a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “selectively” or “selective” may encase a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encases a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In some implementations, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. An infusion line gas removal device, comprising:
a gas permeable tubing coupled between first and second couplers; and
an impermeable tubing shield encasing the gas permeable tubing between the first and second couplers,
wherein the impermeable tubing shield is larger in diameter than the gas permeable tubing and comprises at least one vent and a plurality of supports, the plurality of supports being between the tubing shield and the gas permeable tubing and configured to support the gas permeable tubing within the impermeable tubing shield in a fixed position between the first and second couplers and facilitate passage of gas from the gas permeable tubing to the at least one vent.

2. The infusion line gas removal device of claim 1, wherein the plurality of supports being configured to support the gas permeable tubing within the impermeable tubing shield in a fixed position comprises the plurality of supports being positioned to provide support to an exterior of the gas permeable tubing to prevent a pressure spike within the gas permeable tubing from causing an expansion of the gas permeable tubing.

3. The infusion line gas removal device of claim 2, wherein each of the plurality of supports comprises at least a portion of a disc with an aperture at its center that at least partially encases and provides support to a portion of the gas permeable tubing while facilitating passage of the gas from the gas permeable tubing to the at least one vent, the disc coupled to the impermeable tubing shield.

4. The infusion line gas removal device of claim 3, wherein each of the plurality of supports comprises a gap in an outer edge of the disc between the impermeable tubing shield and the disc.

5. The infusion line gas removal device of claim 4, wherein each gap of the plurality of supports is aligned with the at least one vent.

6. The infusion line gas removal device of claim 1, further comprising:
a bubble diverter coupled to one of the first and second couplers within the gas permeable tubing, the bubble diverter configured to direct bubbles within a fluid flowing inside the gas permeable tubing toward a wall of the gas permeable tubing.

7. The infusion line gas removal device of claim 1, wherein the impermeable tubing shield has an ovoid cross section and the gas permeable tubing has a circular cross section.

8. The infusion line gas removal device of claim 1, wherein one of the first and second couplers comprises an anti-siphon valve configured to facilitate fluid moving in only one direction and to facilitate positive pressure within the gas permeable tubing.

9. The infusion line gas removal device of claim 1, further comprising:
a vacuum source fluidically sealed to the at least one vent, wherein the vacuum source creates a vacuum within the impermeable tubing shield, facilitating removal of the gas from a fluid flowing inside the gas permeable tubing.

10. The infusion line gas removal device of claim 9, wherein the vacuum source comprises:
a vacutainer connected to the at least one vent; and
a flexible membrane, within the vacutainer, configured to flex and become concave within the flexible membrane in response to the vacuum.

11. The infusion line gas removal device of claim 1, wherein the gas permeable tubing comprises:
a pleated interior surface with a plurality of ridges formed along a length of the gas permeable tubing and having a larger surface area than a tubing of a same diameter as the gas permeable tubing.

12. The infusion line gas removal device of claim 1, wherein the gas permeable tubing has a circular cross section at the first and second couplers and an ovoid cross section between the first and second couplers.

13. A process of providing an infusion line gas removal device, comprising:
providing a gas permeable tubing coupled between first and second couplers; and
encasing the gas permeable tubing with an impermeable tubing shield between the first and second couplers,
wherein the impermeable tubing shield is larger in diameter than the gas permeable tubing and comprises at least one vent and a plurality of supports, the plurality of supports being between the tubing shield and the gas permeable tubing and configured to support the gas permeable tubing within the impermeable tubing shield in a fixed position between the first and second couplers and facilitate passage of gas from the gas permeable tubing to the at least one vent.

14. The process of claim 13, further comprising:
forming the plurality of supports within the impermeable tubing shield by positioning the plurality of supports in a manner to provide support to an exterior of the gas permeable tubing to prevent a pressure spike within the gas permeable tubing from causing an expansion of the gas permeable tubing.

15. The process of claim 14, wherein forming the plurality of supports within the impermeable tubing shield comprises:
forming the impermeable tubing shield in two molded cross sections, each cross section traversing a length of a gas permeable tubing and comprising a plurality of partial supports that, when the two molded cross sections are coupled together around the gas permeable tubing, come together to form the plurality of supports and support the gas permeable tubing within the impermeable tubing shield in the fixed position; and
coupling the two molded cross sections around the gas permeable tubing.

16. The process of claim 15, wherein the impermeable tubing shield, when formed, has a circular cross section, and the plurality of supports, when formed, each comprise a disc with an aperture at its center that constrains a portion of the gas permeable tubing.

17. The process of claim 14, wherein each of the plurality of supports comprises at least a portion of a disc with an aperture at its center that at least partially encases and provides support to a portion of the gas permeable tubing while facilitating passage of the gas from the gas permeable tubing to the at least one vent, the disc coupled to the impermeable tubing shield.

18. The process of claim 13, further comprising:
fluidically sealing a vacuum source to the at least one vent, wherein the vacuum source creates a vacuum within the impermeable tubing shield, facilitating removal of the gas from a fluid flowing inside the gas permeable tubing.

19. A method for removal of gas from an infusion line, comprising:
providing a gas permeable tubing coupled between first and second couplers; and
providing an impermeable tubing shield encasing the gas permeable tubing between the first and second couplers,
wherein the impermeable tubing shield is larger in diameter than the gas permeable tubing and comprises at least one vent and a plurality of supports, the plurality of supports being between the tubing shield and the gas permeable tubing and configured to support the gas permeable tubing within the impermeable tubing shield in a fixed position between the first and second couplers and facilitate passage of gas from the gas permeable tubing to the at least one vent.

20. The method of claim 19, further comprising:
providing a vacuum source configured to be fluidically sealed to the at least one vent, wherein the vacuum source creates a vacuum within the impermeable tubing shield, facilitating removal of the gas from a fluid flowing inside the gas permeable tubing, and includes a flexible membrane configured to flex and become concave within the flexible membrane in response to the vacuum.
